(19) United States
(12) Patent Application Publication
(10) Pub. No.: US 2022/0036281 A1
(43) Pub. Date: Feb. 3, 2022

(54) Evaluating medical providers

(71) Applicant: Spring Care, Inc., New York, NY (US)

(72) Inventors: Adam Chekroud, New York, NY (US); Kaelen Medeiros, Astoria, NY (US); Kelsey Quick, Spring Lake, NJ (US)

(21) Appl. No.: 17/389,702

(22) Filed: Jul. 30, 2021

Related U.S. Application Data
(60) Provisional application No. 63/058,765, filed on Jul. 30, 2020.

(51) Int. Cl. G06Q 10/06; G16H 40/20; G06N 20/00

(52) U.S. Cl. CPC G06Q 10/06398; G06Q 10/06395; G06N 20/00; G16H 40/20; G06Q 10/063112

(57) ABSTRACT

Methods and systems for evaluating one or more medical providers. The methods include receiving data associated with each of the one or more providers, executing a trained evaluation model to calculate a quality score for each of the one or more providers based on the received data, and performing at least one of: recommending at least one provider to treat a patient based on the calculated quality score for each of the one or more providers, or detecting that a calculated quality score for a provider fails to meet a treatment threshold.

### CROSS-REFERENCE TO RELATED APPLICATIONS

This application claims priority to and the benefit of U.S. Provisional Patent Application No. 63/058,765, filed on Jul. 30, 2020, which is hereby incorporated by reference in its entirety.

### TECHNICAL FIELD

Embodiments described herein generally relate to systems and methods for providing healthcare and, more particularly but not exclusively, to systems and methods for analyzing the efficacy of healthcare providers.

### BACKGROUND

Matching healthcare providers and patients is a challenging endeavor. There are several factors or aspects of the provider-patient relationship that may impact whether a certain provider is an appropriate match for a patient. Similarly, there are several factors that may affect a particular provider's efficacy in providing treatment to a particular patient. A patient's insurance coverage may further limit the number of providers that are available for a patient.

There are also no convenient methodologies for quantifying, measuring, or otherwise evaluating a provider's performance. Even if a provider has successfully treated a patient, there is no guarantee or way to predict whether they will be an appropriate match for another patient with the same, similar, or different ailment. Instances of unsuccessful treatment may also go unnoticed such that a provider or healthcare institution may be unaware of the treatment failure or the need to change treatment methodologies in the future.

A need exists, therefore, for systems and methods for evaluating one or more medical providers to most appropriately match providers with patients.

### SUMMARY

This summary is provided to introduce a selection of concepts in a simplified form that are further described below in the Detailed Description section. This summary is not intended to identify or exclude key features or essential features of the claimed subject matter, nor is it intended to be used as an aid in determining the scope of the claimed subject matter.

In one aspect, embodiments relate to a method for evaluating one or more medical providers. The method includes receiving data associated with each of the one or more providers, executing a trained evaluation model to calculate a quality score for each of the one or more providers based on the received data, and performing at least one of recommending at least one provider to treat a patient based on the calculated quality score for each of the one or more providers, or detecting that a calculated quality score for a provider fails to meet a treatment threshold.

In some embodiments, the method further includes executing a machine learning procedure on the received data to derive the treatment threshold.

In some embodiments, the method further includes generating a plurality of treatment thresholds that are each associated with a quality metric, comparing the received data to the plurality of treatment thresholds, and determining a number of treatment thresholds satisfied by data associated with a provider of the one or more providers, wherein the quality score for the provider is based on the number of treatment thresholds satisfied by the data associated with the provider. In some embodiments, the method further includes, using the classifier, awarding a point for each treatment threshold satisfied by the data associated with the provider. In some embodiments, assigning a weight to each of the at least one quality metric, wherein the weight assigned to each of the at least one quality metric is based on how relevant the quality metric is in providing treatment to a patient.

In some embodiments, the method further includes executing a machine learning procedure on the received data to identify at least one quality metric that is relevant in calculating the quality score.

In some embodiments, the method further includes elevating a provider for coaching upon detecting that the calculated quality score for the provider fails to meet the treatment threshold.

In some embodiments, the data is received from scheduling software, billing software, or records of a patient associated with a provider.

According to another aspect, embodiments relate to a system for evaluating one or more medical providers. The system includes an interface for at least receiving data associated with each of the one or more providers; and a processor executing instructions stored on a memory and configured to execute a trained evaluation model to calculate a quality score for each of the one or more providers based on the received data, and at least one of recommending at least one provider to treat a patient based on the calculated quality score for each of the one or more providers, or detecting that a calculated quality score for a provider fails to meet a treatment threshold.

In some embodiments, the processor is further configured to execute a machine learning procedure on the received data to derive the treatment threshold.

In some embodiments, the processor is further configured to generate a plurality of treatment thresholds that are each associated with a quality metric, compare the received data to the plurality of treatment thresholds, and determine a number of treatment thresholds satisfied by data associated with a provider of the one or more providers, wherein the quality score for the provider is based on the number of treatment thresholds satisfied by the data associated with the provider. In some embodiments, the processor is further configured to award a point for each treatment threshold satisfied by the data associated with the provider. In some embodiments, the processor is further configured to assign a weight to each of the at least one quality metric, wherein the weight assigned to each of the at least one quality metric is based on how relevant the quality metric is in providing treatment to a patient.

In some embodiments, the processor is further configured to execute a machine learning procedure on the received data to identify at least one quality metric that is relevant in calculating the quality score.

In some embodiments, the processor is further configured to elevate a provider for coaching upon detecting that the calculated quality score for the provider fails to meet the treatment threshold.

In some embodiments, the data is received from scheduling software, billing software, or records of a patient associated with a provider.

According to yet another aspect, embodiments relate to a non-transitory computer readable storage medium containing computer-executable instructions for evaluating one or more medical providers. The storage medium includes computer-executable instructions for receiving data associated with each of the one or more providers, computer-executable instructions for executing a trained evaluation model to calculate a quality score for each of the one or more providers based on the received data, and computer-executable instructions for performing at least one of recommending at least one provider to treat a patient based on the calculated quality score for each of the one or more providers, or detecting that a calculated quality score for a provider fails to meet a treatment threshold.

In some embodiments, the medium further includes computer-executable instructions for executing a machine learning procedure on the received data to derive the treatment threshold.

In some embodiments, the medium further includes computer-executable instructions for generating a plurality of treatment thresholds that are each associated with a quality metric, computer-executable instructions for comparing the received data to the plurality of treatment thresholds, and computer-executable instructions for determining a number of treatment thresholds satisfied by data associated with a provider of the one or more providers, wherein the quality score for the provider is based on the number of treatment thresholds satisfied by the data associated with the provider. In some embodiments, the medium further includes computer-executable instructions for awarding a point for each treatment threshold satisfied by the data associated with the provider.

### BRIEF DESCRIPTION OF DRAWINGS

Non-limiting and non-exhaustive embodiments of the invention are described with reference to the following figures, wherein like reference numerals refer to like parts throughout the various views unless otherwise specified.

FIG. 1 illustrates a system for evaluating one or more medical providers in accordance with one embodiment;

FIG. 2 illustrates the processor of FIG. 1 in accordance with one embodiment;

FIG. 3 presents a table of exemplary metrics and standards related to a provider's treatment of one or more patients in accordance with one embodiment; and

FIG. 4 depicts a flowchart of a method for evaluating one or more medical providers in accordance with one embodiment.

### DETAILED DESCRIPTION

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment. The appearances of the phrase “in some embodiments” in various places in the specification are not necessarily all referring to the same embodiments.

Some portions of the description that follow are presented in terms of symbolic representations of operations on non-transient signals stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to these signals as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

The systems and methods described herein enable measuring or otherwise monitoring the performance of clinicians, medical providers, administrators, licensed practitioners, or the like (for simplicity, “provider(s)”) in real-time. The embodiments described herein may track patient-level clinical outcomes and provider-level data, and leverage continuous feedback to ensure that patients are matched with the best providers. The systems and methods also analyze and evaluate the quality of care provided by providers based on streaming data obtained and in real time.

This is in contrast to existing methodologies for monitoring provider performance. Existing methodologies involve storing data for some period of time, creating reports based on the stored data, and then eventually having operational teams review said reports. Accordingly, these existing methodologies are inherently retroactive and do not provide ways to monitor and improve provider performance in real-time.

The systems and methods described herein may analyze clinical outcomes and adherence to procedural duties to evaluate the care provided by individual providers. The results may be used to determine when providers fail to meet quality thresholds, determine when providers meet or exceed quality thresholds, offer “pay for performance” incentives, rank providers, match patients and providers, or the like. Additionally, the results may help formalize a model for defining criteria (e.g., through machine learning) and objective metrics to help improve the quality of provider care. In some embodiments, the results of the analyses performed by the systems and methods herein may be used for making hiring decisions.

The embodiments herein may integrate data from multiple sources related to patient treatment. Using this data, the described embodiments may calculate machine-learned quality scores to assess providers' effectiveness. For example, the calculated quality scores may provide insight into a provider's clinical efficacy, financial efficacy, and whether they will be a successful match for a patient.

As used in the present application “provider/s” may also refer to staff such as care coordinators, care navigators, counselors, therapists, peer supporters, social workers, operational staff, administrative staff, etc., or any other personnel involved in providing some type of treatment to individuals. This treatment may involve therapy or medication management, for example. In addition to or in lieu of providers, the systems and methods herein are applicable to provider groups, healthcare systems, healthcare institutions, health insurance companies, or any other type of entity that operates with or otherwise utilizes health provider networks.

The systems and methods described herein may track a variety of information at the patient and provider levels through various methods. These methods may include, but are not limited to, patient-facing measurement platforms, provider level data feeds, or the like. The embodiments described herein may integrate multiple data types in real time, calculate performance measures and metrics concerning one or more providers, and calculate machine-learned quality measures to rank providers according to their clinical and financial quality.

These rankings may also be based on a likelihood a particular provider would be an appropriate match for a specific patient. These rankings may be based on data available entirely from actions taken by prospective patients and providers before the booking of an appointment (i.e., before a provider delivers care and before a patient receives care). This is in contrast to existing techniques, which as discussed above are inherently retroactive and can only be performed weeks, months, or years after an observable event.

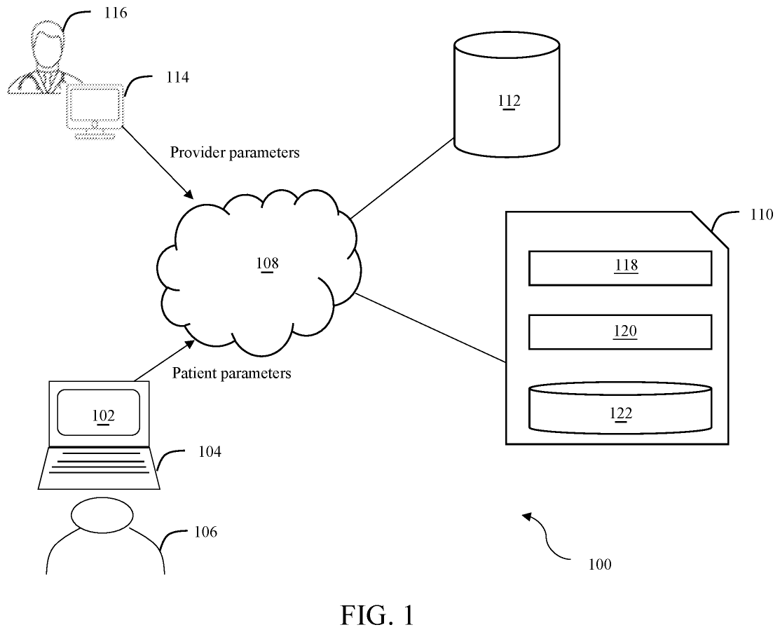

FIG. 1 illustrates a system 100 for evaluating one or more medical providers in accordance with one embodiment. The system 100 may include a user interface 102 executing on a user device 104 to at least enable a patient 106 to provide feedback related to their treatment experience.

The user device 104 may be connected to or otherwise in operable communication with a network 108 and be in further communication with a server 110, one or more databases 112 storing the patient's electronic medical record (EMR), and a provider device 114 for use by a provider 116.

The user interface 102 may allow the patient 106 to input parameters related to their background, health, and treatment. For example, the user interface 102 may present a questionnaire to the patient 106 prompting the patient 106 to provide certain data. If the patient 106 suffers from depression and anxiety, the provided information may include whether they believe their depression has improved from treatment, whether their anxiety has improved, whether they have adhered to prescribed medication or a treatment regime, or the like.

The user interface 102 may be configured as a responsive web application frontend or a native mobile application. The application may communicate with the server 110 over SSL to ensure encryption of all patient-related data in transit.

The user device 104 may be any suitable input/output device that can execute the user interface 102. The user device 104 may be configured as a PC, a laptop, a tablet, a smartwatch, a smartphone, a kiosk, or the like. The user device 104 may be a personal device (e.g., located at the patient's home), or may be located at the provider's office. The exact configuration or implementation of the user device 104 may vary as long as the features of various embodiments described herein can be accomplished.

Additionally, patient data and responses to any questionnaires may be cached on the user device 104 (e.g., either in secure storage on iOS, or signed cookies in a web browser of the patient's personal user device). This ensures that patient data is stored securely, and that the data is cached on the user device 104 until a connection is established between the user device 104 and the server 110. Additionally, the data may be cached on the user device 104 until the connection between the user device 104 and the server 110 is stable.

If the user device 104 is located in a provider's office or otherwise a public space, the cache may be invalidated every time a new patient starts using the user device 104. Or, in the case of a personal device, the cache may be invalidated when the patient logs out of their own device. This ensures that a patient's information is only cached during an active session.

Although only one patient 106 is shown in FIG. 1, the embodiments herein may simultaneously receive patient parameters from a plurality of patients. This data can be received and analyzed in real time to gain more up-to-date and accurate information.

The network(s) 108 may link the various components with various types of network connections. The network(s) 108 may be comprised of, or may interface to, any one or more of the Internet, an intranet, a Personal Area Network (PAN), a Local Area Network (LAN), a Wide Area Network (WAN), a Metropolitan Area Network (MAN), a storage area network (SAN), a frame relay connection, an Advanced Intelligent Network (AIN) connection, a synchronous optical network (SONET) connection, a digital T1, T3, E1, or E3 line, a Digital Data Service (DDS) connection, a Digital Subscriber Line (DSL) connection, an Ethernet connection, an Integrated Services Digital Network (ISDN) line, a dial-up port such as a V.90, a V.34, or a V.34bis analog modem connection, a cable modem, an Asynchronous Transfer Mode (ATM) connection, a Fiber Distributed Data Interface (FDDI) connection, a Copper Distributed Data Interface (CDDI) connection, or an optical/DWDM network.

The network or networks 108 may also comprise, include, or interface to any one or more of a Wireless Application Protocol (WAP) link, a Wi-Fi link, a microwave link, a General Packet Radio Service (GPRS) link, a Global System for Mobile Communication G(SM) link, a Code Division Multiple Access (CDMA) link, or a Time Division Multiple access (TDMA) link such as a cellular phone channel, a Global Positioning System (GPS) link, a cellular digital packet data (CDPD) link, a Research in Motion, Limited (RIM) duplex paging type device, a Bluetooth radio link, or an IEEE 802.11-based link.

The server 110 may include a processor 118 executing instructions stored on a memory 120 to provide an evaluation model. The server 110 may also include a storage 122 for at least storing outputs from the evaluation module.

The processor 118 may be any hardware device capable of executing instructions stored on the memory 120 to provide the evaluation model. The processor 118 may include a microprocessor, a field programmable gate array (FPGA), an application-specific integrated circuit (ASIC), or other similar devices. In some embodiments, such as those relying on one or more ASICs, the functionality described as being provided in part via software may instead be configured into the design of the ASICs and, as such, the associated software may be omitted. The processor 118 may be configured as part of the user device 104 (e.g., a laptop), located at some remote location (such as on the server 110), or part of the provider device 114.

The memory 120 may be L1, L2, L3 cache, or RAM memory configurations. The memory 120 may include non-volatile memory such as flash memory, EPROM, EEPROM, ROM, and PROM, or volatile memory such as static or dynamic RAM, as discussed above. The exact configuration/type of memory 120 may of course vary as long as instructions for evaluating one or providers can be performed the system 100.

The storage 122 may include one or more machine-readable storage media such as read-only memory (ROM), random-access memory (RAM), magnetic disk storage media, optical storage media, flash-memory devices, or similar storage media. In various embodiments, the storage 122 may store instructions for execution by the processor 118 as well as outputs of the evaluation model.

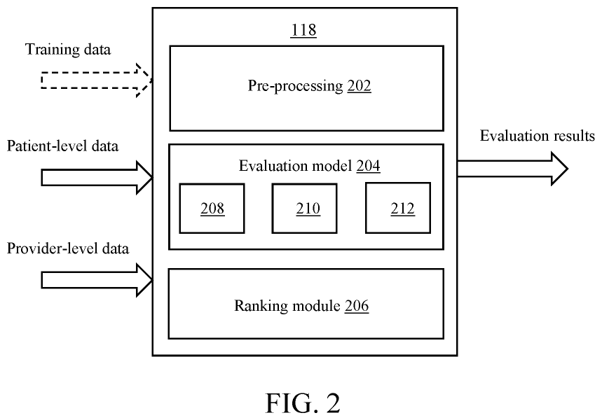

FIG. 2 illustrates the processor 118 of FIG. 1 in more detail. In some embodiments, the processor 118 may receive as input training data, patient-level parameters, and provider-level parameters. The processor 118 may execute a pre-processing module 202, an evaluation model 204, and a ranking module 206.

The training data may comprise data related to previous patients and their treatment. For example, the training data may include established thresholds that should be used for each metric, discussed below. In these embodiments, the evaluation model 204 may execute supervised learning procedures when analyzing patient- and provider-level data.

This training data may include data from a variety of sources. For example, the model 204 may be trained on aggregated data from the U.S. National Survey on Drug Use and Health series, and data collected from adults diagnosed with depression by a healthcare provider in the 12 months prior to the survey. These data sources are merely exemplary, and the evaluation model 204 may be trained on other data sources, whether available now or compiled hereafter.

Training data may not be required in some embodiments. In these embodiments, the evaluation model 204 may execute unsupervised learning procedures on the received data to, for example, determine which metrics are most relevant in evaluating the quality of care, as well as metrics' associated thresholds.

The patient-level data may be collected from patient clinical notes and from patient responses regarding their health. For example, and as discussed previously, patients may be presented with surveys in which they are prompted to answer questions about their health and received treatment.

The provider-level data may be collected from a variety of data sources. These sources may include, but are not limited to, billing systems, surveys, scheduling software, baseline assessment data, follow-up assessment data, provider-taken notes, or the like. As seen in FIG. 1, a provider 116 may also use a provider device 114 to provide data related to their patients and treatment provided.

The pre-processing module 202 may perform any required processing steps on received data. For example, the pre-processing module 202 may calculate various derivatives of received data, including but not limited to simple averages (i.e., a mean(s), weighted averages, standard deviations, etc.). Similarly, the pre-processing module 202 may require that providers have a minimum quantity of associated data (e.g., they have treated a minimum number of patients) to ensure that outliers do not skew results.

The evaluation model 204 may execute supervised or unsupervised machine learning procedures to analyze the received data and calculate quality scores associated with the providers. The evaluation model 204 may execute a weighting component 208, a point component 210, and a quality score component 212.

The weighting component 208 may assign weight(s) to each metric based on the relative importance of the metric. For example, the weighting component 208 may execute machine learning procedures to determine the relative weights and combinations of quality metrics across that best evaluate providers.

The point component 210 may award points to a provider based on the provider satisfying one or more thresholds. For example, for each metric a provider satisfies, the point component 210 may award a point.

The quality score component 212 may assign a quality score to each analyzed provider based on their associated data. In the context of the present application, a “quality score” may represent the efficacy of the provider's treatment choices with respect to the provider's patients. The quality score may be based on a plurality of factors or metrics related to the provider-patient relationship and treatment. For example, the quality score component 212 may aggregate all points awarded to a provider by the point component 210 to generate the quality score.

These metrics may be based on previously-observed data, clinical guidelines, gold standards, evidence from practice, and historical data associated with a particular provider or healthcare institution. Metrics related to a patient suffering from depression may include whether a patient has reported an improvement in clinical depression, whether a patient has reported an improvement in clinical anxiety, whether a patient has reported a remission in anxiety, patient adherence to prescribed medication or treatment, or some combination thereof. Metrics related to a provider in treating a patient suffering from depression may include their note quality or completion, their appointment consistency (e.g., whether they have cancelled any appointments), their appointment availability, or some combination thereof.

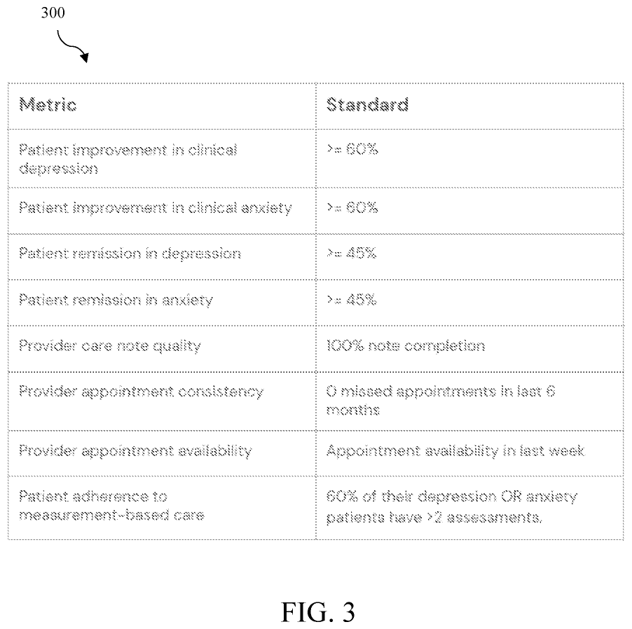

Each metric may be associated with some threshold value that indicates a minimum level of quality of care provided or treatment success. For example, FIG. 3 presents a table 300 of exemplary metrics and standards related to a provider's treatment of one or more patients. As seen in the table 300, analyzed metrics listed in the “Metric” column include those discussed above. The table 300 also includes a “Standard” column that specifies threshold values associated with each associated metric.

The evaluation model 204 may, for each provider, determine whether the provider's associated data satisfies the thresholds of one or more of the listed metrics and award point(s) for each satisfied threshold. For example, a provider may be awarded one point if they haven't missed or cancelled an appointment in the last six months. As another example, a provider may be awarded a point if 60% or more of their patients reported an improvement in clinical anxiety.

Some metrics may be worth more than others. In these scenarios, the point component 210 may award more than one point to a provider for satisfying a particular metric's associated threshold. A provider may also be awarded additional points based on the amount by which they exceed a threshold. For example, a provider may be awarded one point if 60-69% of their patients reported an improvement in clinical anxiety, two points if 70-79% of their patients reported an improvement in clinical anxiety, etc.

The quality metrics of FIG. 3 are exemplary, and other metrics and other types of treatment may be considered. The type of treatment(s) considered may depend on the ailments or condition of a particular patient.

Additionally, a single provider may provide multiple kinds of treatment. The embodiments described herein may therefore rank a specific provider based on how well they provide a certain type of treatment. For talk-related therapy, for example, the embodiments herein may consider treatment such as medication prescribed, residential programs, peer support provided, or the like.

The ranking module 206 may rank each provider based on their calculated quality scores. For example, the ranking module 206 may output evaluation results in the form of a list that ranks each provider based on their quality score.

These techniques can also help drive down costs over time by selectively boosting or ranking providers in the database 112 or scheduling platforms based on their financial effectiveness (e.g., their “cost per remission”). Similarly, these techniques can be used to pay providers based on demonstrated, objectively-measured clinical values at the patient level. The calculated metrics can also be the basis for upside or downside clinical risk sharing agreements.

The embodiments herein may initially be configured with or without the thresholds such as those in FIG. 3. In these embodiments, the systems and methods herein may statistically learn thresholds to ensure the metrics ultimately used are causal and optimal, and also to ensure that the combination or weighting of the used metrics are optimal.

The calculated metrics and points may be provided to an administrator in a dashboard and/or stored in the database 112 for further analysis. This data may be made available to multiple parties via a representational state transfer (REST) API or another API architecture. Additionally, and as discussed above, the data points can be streamed live and in real time to facilitate business operations.

Administrators are therefore able to track how providers are performing over time. A number of different actions may be performed based on the evaluation results. For example, scheduling procedures may in the future direct more patients to providers with high calculated metrics and resultant rankings.

As another example, institutions may incentivize providers with higher salaries or “pay-per-performance” incentives based on calculated metrics and rankings. Additionally or alternatively, institutions may use the evaluations to coach and train providers in areas where they are performing well and areas where they are performing poorly against their peers.

When a provider fails to meet established thresholds, administrators can intervene with coaching to help providers treat their patients more effectively. The results of the analyses may be made available to human resource personnel, clinical operations teams, or anyone else involved in an evaluation process. In addition to or in lieu of purely “evaluation” purposes, the embodiments herein may help providers obtain quantitative insights into their case load, individual patient progress, overall clinical or financial effectiveness, impact of a Qualified Individual (QI) program, or the like.

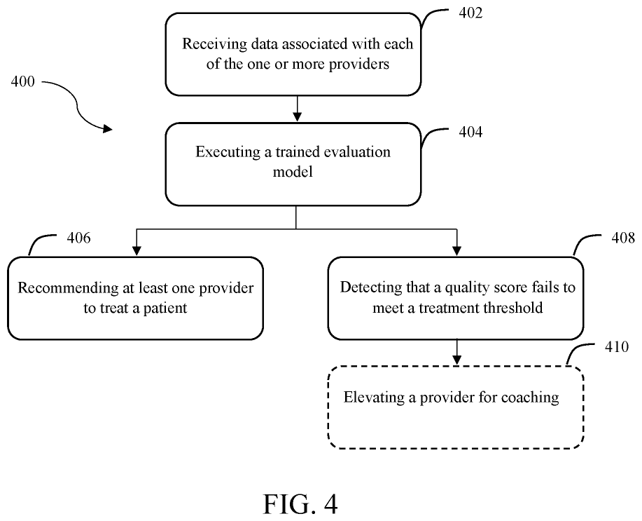

FIG. 4 depicts a flowchart of a method 400 for evaluating one or more medical providers in accordance with one embodiment. The system 100 of FIG. 1 or components thereof may perform the steps of method 400.

Step 402 involves receiving data associated with each of the one or more providers. This data may be received from sources such as, but not limited to, scheduling software, billing software, or records of a patient associated with a provider.

Step 404 involves executing a trained evaluation model to calculate a quality score for each of the one or more providers based on the received data. The evaluation model may be trained on the received data, in which case the evaluation model executes unsupervised machine learning procedures. Additionally or alternatively, the evaluation model may be previously trained on training data.

The calculated quality score(s) may be based on whether a provider has satisfied certain metric(s) related to patient treatment. For example, the more metrics that a provider satisfies, the higher their quality score will be.

As discussed above, points may be awarded to a provider for each treatment threshold the provider satisfies. The model may also assign weights to the metrics based on how relevant the metric is in treating a patient. In some embodiments, the calculated quality score may be the sum of all points awarded for satisfying the applicable metrics.

Step 406 involves recommending at least one provider to treat a patient based on the calculated quality score for each of the one or more providers. In some embodiments, providers who demonstrate a certain level or quality of care may be recommended for treatment of new patients. For example, if a provider has a demonstrated record of successfully treating patients with specific conditions, they may be more likely to be recommended to treat new patients with the same condition(s).

Step 408 involves detecting that a calculated quality score for a provider fails to meet a treatment threshold. In some instances, the embodiments described herein may detect if and when a quality score associated with a provider fails to meet a threshold. A low-quality score may be the result of the provider failing to meet a number of treatment thresholds, for example. In these cases, the provider may be informed of their low-quality score, and an administrator of a healthcare institution may be informed as well. For example, step 410 is optional and involves elevating a provider for coaching upon detecting their quality score fails to meet a treatment threshold.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

What is claimed is:

1. A method for evaluating one or more medical providers, the method comprising:
receiving data associated with each of the one or more providers;
executing a trained evaluation model to calculate a quality score for each of the one or more providers based on the received data; and
performing at least one of:
recommending at least one provider to treat a patient based on the calculated quality score for each of the one or more providers, or
detecting that a calculated quality score for a provider fails to meet a treatment threshold.

2. The method of claim 1 further comprising executing a machine learning procedure on the received data to derive the treatment threshold.

3. The method of claim 1 further comprising:
generating a plurality of treatment thresholds that are each associated with a quality metric,
comparing the received data to the plurality of treatment thresholds, and
determining a number of treatment thresholds satisfied by data associated with a provider of the one or more providers,
wherein the quality score for the provider is based on the number of treatment thresholds satisfied by the data associated with the provider.

4. The method of claim 3 further comprising, using the classifier, awarding a point for each treatment threshold satisfied by the data associated with the provider.

5. The method of claim 4 further comprising assigning a weight to each of the at least one quality metric, wherein the weight assigned to each of the at least one quality metric is based on how relevant the quality metric is in providing treatment to a patient.

6. The method of claim 1 further comprising executing a machine learning procedure on the received data to identify at least one quality metric that is relevant in calculating the quality score.

7. The method of claim 1 further comprising elevating a provider for coaching upon detecting that the calculated quality score for the provider fails to meet the treatment threshold.

8. The method of claim 1 wherein the data is received from scheduling software, billing software, or records of a patient associated with a provider.

9. A system for evaluating one or more medical providers, the system comprising:
an interface for at least receiving data associated with each of the one or more providers; and
a processor executing instructions stored on a memory and configured to:
execute a trained evaluation model to calculate a quality score for each of the one or more providers based on the received data, and at least one of:
recommending at least one provider to treat a patient based on the calculated quality score for each of the one or more providers, or
detecting that a calculated quality score for a provider fails to meet a treatment threshold.

10. The system of claim 9 wherein the processor is further configured to execute a machine learning procedure on the received data to derive the treatment threshold.

11. The system of claim 9 wherein the processor is further configured to:
generate a plurality of treatment thresholds that are each associated with a quality metric,
compare the received data to the plurality of treatment thresholds, and
determine a number of treatment thresholds satisfied by data associated with a provider of the one or more providers, wherein the quality score for the provider is based on the number of treatment thresholds satisfied by the data associated with the provider.

12. The system of claim 11 wherein the processor is further configured to award a point for each treatment threshold satisfied by the data associated with the provider.

13. The system of claim 12 wherein the processor is further configured to assign a weight to each of the at least one quality metric, wherein the weight assigned to each of the at least one quality metric is based on how relevant the quality metric is in providing treatment to a patient.

14. The system of claim 9 wherein the processor is further configured to execute a machine learning procedure on the received data to identify at least one quality metric that is relevant in calculating the quality score.

15. The system of claim 9 wherein the processor is further configured to elevate a provider for coaching upon detecting that the calculated quality score for the provider fails to meet the treatment threshold.

16. The system of claim 9 wherein the data is received from scheduling software, billing software, or records of a patient associated with a provider.

17. A non-transitory computer readable storage medium containing computer-executable instructions for evaluating one or more medical providers, the medium comprising:
computer-executable instructions for receiving data associated with each of the one or more providers;
computer-executable instructions for executing a trained evaluation model to calculate a quality score for each of the one or more providers based on the received data; and
computer-executable instructions for performing at least one of:
recommending at least one provider to treat a patient based on the calculated quality score for each of the one or more providers, or
detecting that a calculated quality score for a provider fails to meet a treatment threshold.

18. The non-transitory computer readable medium of claim 17 further comprising computer-executable instructions for executing a machine learning procedure on the received data to derive the treatment threshold.

19. The non-transitory computer readable medium of claim 17 further comprising:
computer-executable instructions for generating a plurality of treatment thresholds that are each associated with a quality metric,
computer-executable instructions for comparing the received data to the plurality of treatment thresholds, and
computer-executable instructions for determining a number of treatment thresholds satisfied by data associated with a provider of the one or more providers,
wherein the quality score for the provider is based on the number of treatment thresholds satisfied by the data associated with the provider.

20. The non-transitory computer readable medium of claim 19 further comprising computer-executable instructions for awarding a point for each treatment threshold satisfied by the data associated with the provider.
